(19) United States
(12) Patent Application Publication
(10) Pub. No.: US 2017/0300619 A1
(43) Pub. Date: Oct. 19, 2017

(54) MEDICAL IMAGING SYSTEM

(71) Applicant: ACIST MEDICAL SYSTEMS, INC., Eden Prairie, MN (US)

(72) Inventor: Adam Rondeau, Arden Hills, MN (US)

(21) Appl. No.: 15/489,935

(22) Filed: Apr. 18, 2017

Related U.S. Application Data
(60) Provisional application No. 62/324,722, filed on Apr. 19, 2016.

(51) Int. Cl. G06F 19/00; A61M 5/00; A61B 6/00; A61B 8/12; A61M 25/01

(52) U.S. Cl. CPC G06F 19/321; G06F 19/322; A61M 25/01; A61B 6/504; A61B 8/12; A61M 5/007

(57) ABSTRACT

A system for transferring patient identification information and medical images within the records system of a healthcare facility is disclosed. A server within the healthcare facility is configured to automatically receive patient information on a regular basis from the healthcare facility's patient information system and to automatically provide that information to any medical equipment connected to the healthcare facility. The operator of the medical equipment selects the information of a patient undergoing a medical imaging procedure. The resulting medical image is associated with the chosen patient. The server disclosed herein receives the medical image and the patient identification information from the medical equipment, and automatically converts the medical image into a standardized file format. The system automatically transfers the converted medical image and patient identification information to the healthcare facility's medical image storage system, which stores the information.

### CLAIM OF PRIORITY

This application claims the benefit of priority to U.S. Provisional Patent Application No. 62/324,722, filed on Apr. 19, 2016 and entitled, “MANAGEMENT OF MEDICAL IMAGE INFORMATION,” the entire contents of which is incorporated herein by reference in its entirety.

### TECHNICAL FIELD

This document relates generally to the operation of a system for transferring and storing medical records.

### BACKGROUND

Medical information storage systems are used in many healthcare facilities to store patient medical information. One example is medical image information, which must be adequately and accurately associated with an individual patient before it is archived.

In many healthcare facilities, a database of detailed, personally identifiable patient information is maintained. Medical imaging equipment is also maintained, which allows the facility to run procedures and produce medical images and associated data. In many facilities, the database of patient identification information is not readily accessible by the medical imaging equipment. Consequently, the operator of medical imaging equipment must manually collect and input patient identification information before running a medical procedure. This time-consuming data entry is often necessary to ensure that the medical image produced by the equipment is associated with a specific patient.

Additionally, because the database of patient identification information and the medical imaging equipment are often not integrated into the medical information system of the healthcare facility, medical image information is not accessible to employees other than the operator of the medical equipment. To make medical image information broadly available, manual entry is necessary. This lack of connectivity means that the operator of medical imaging equipment must input necessary patient information, and makes it more difficult for other employees to review, input, or correct patient identification information associated with a medical image. And manual entry can increase the likelihood of error.

Medical image information must also be stored in a specific system, such as a picture archiving and communication system (“PACS”). Medical images and associated data must be converted into a specific format for storage, such as the Digital Imaging and Communications in Medicine (“DICOM”) standard. Typically, the tasks of converting medical images into DICOM and other similar formats and storing the converted data in the PACS or similar system must be performed by staff of the healthcare facility.

### SUMMARY

Embodiments of the present invention automatically transfer information within the system of a healthcare facility. Some embodiments allow medical equipment to electronically link with existing information systems of a healthcare facility. Medical equipment, including equipment used in medical imaging procedures, may automatically receive patient information from healthcare facility systems via a server. Information generated by the medical equipment may be automatically transferred to healthcare facility systems via a server, which may process the information prior to transfer. Automating the file transfer process can help to save time and promote accurate record keeping. This in turn can streamline the process of performing medical procedures. Automatic conversion of files outside of existing healthcare facility systems can reduce the amount of space healthcare facility systems need to transmit, store, and process files. Automation of these processes can therefore be desirable as a means of saving time, streamlining medical procedures, and improving the accessibility and reliability of patient records.

### BRIEF DESCRIPTION OF DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1 is a schematic diagram of a healthcare facility using an information transfer system.

FIG. 2 is flowchart depicting the processing of information in an information transfer system.

FIG. 3 is an example of a patient data set.

FIG. 4 is an example of a display indicating the storage status of information.

FIG. 5 is an example of a display indicating the conversion and storage status of information.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention. Examples of constructions, materials, dimensions, and manufacturing processes may be provided for selected elements, and all other elements employ that which is known to those of skill in the field of the invention. Those skilled in the art will recognize that many of the examples provided have suitable alternatives that can be utilized.

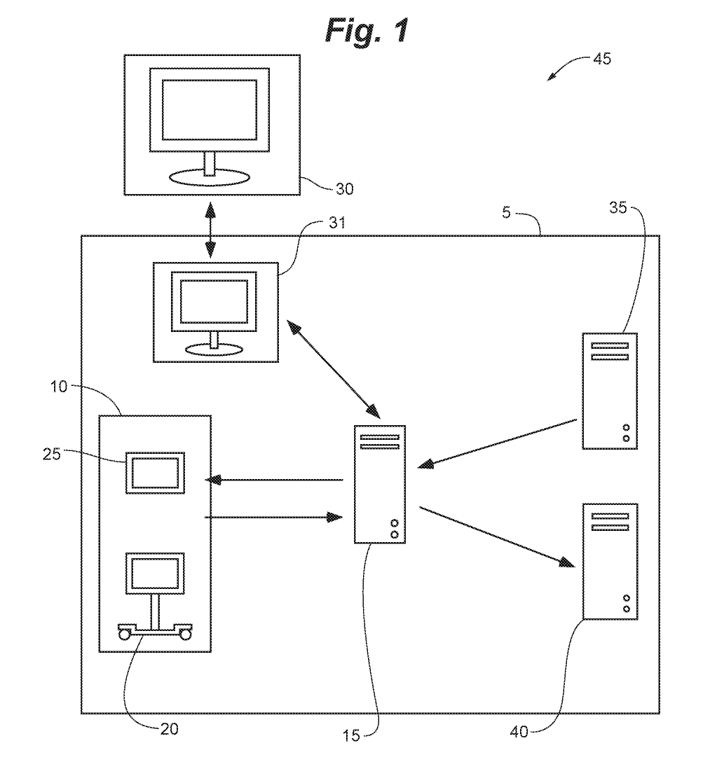

FIG. 1 is a schematic representation of a healthcare facility 5. The technology described in the present disclosure may be used in a wide variety of settings. Modern healthcare entities include not only traditional hospital clinics, but also specialty centers, distributed campuses, and telecommunication-based practices, among other examples. In some embodiments, healthcare facility 5 may comprise a hospital. Healthcare facility 5 may comprise a medical clinic. Healthcare facility 5 may comprise a department. Healthcare facility 5 may comprise several medical establishments that share certain systems.

Healthcare facility 5 may include medical examination room 10 in which medical procedures may be performed on patients. Healthcare facility 5 may include various medical examination rooms 10. In some embodiments, medical examination room 10 may comprise a room for a specific procedure. Medical examination room 10 may comprise a catheterization laboratory. Medical examination room 10 may comprise a radiology examination room.

Healthcare facility 5 may include various computing devices for sending and receiving information among each other within healthcare facility 5. In many instances, computing devices may send and receive information from sources outside of healthcare facility 5.

In some embodiments, healthcare facility 5 includes or utilizes a server 15 that facilitates communication among the other computing devices. Server 15 may be located outside of medical examination room 10, typically within healthcare facility 5. In some embodiments, server 15 includes a computer. In some embodiments, server 15 includes a local computer network. According to some embodiments, server 15 includes a remote computer and/or a remote computer network.

Healthcare facility 5 may include medical equipment 20 for performing various procedures on patients. Medical equipment 20 may be located in medical examination room 10, which may be in a different location than server 15. According to some embodiments, medical equipment 20 includes a medical imaging device. A medical imaging device may include an intravascular ultrasound system. A medical imaging device may include a radiology system. In some embodiments, the medical equipment 20 can include a contrast injection system. In some embodiments, the medical equipment 20 can include a fractional flow reserve measurement system. Various other kinds of medical equipment (e.g., hemodynamic devices, angiographic devices, heartrate monitors, blood gas analyzers, etc.) can be used in systems as described in this disclosure.

In some embodiments, medical equipment 20 includes a console display 25. As persons familiar with the art will realize, a console display 25 may be connected to medical equipment 20 in multiple ways, including wired and/or wireless connections (including Bluetooth), or any other method of connecting medical equipment to a display. Console display 25 may be located on a medical imaging device. Console display 25 may be located on a monitor connected to a medical imaging device.

Healthcare facility 5 may include one or more display devices 30, 31 that enable a user to view medical information in a variety of formats. Display device 30 may be located outside of healthcare facility 5. Display device 31 may be located within the healthcare facility 5 and may be locally connected to server 15. Display device 30, 31 may include any device on which a user may use an Internet browser to connect to elements of healthcare facility 5, such as on a computer, tablet, or smartphone. Display device 30, 31 may be located outside of medical examination room 10. Display device 30, 31 may be in a different location than one or more elements of healthcare facility 5. In some embodiments, server 15 provides a web-based interface that allows users with sufficient authorization (e.g., system administrators and technicians) to manage (e.g., review, reconcile, update, delete, etc.) studies and settings from any computer or device that has an Internet browser.

In some embodiments, healthcare facility 5 includes or utilizes a patient information system 35. Patient information system 35 may be located in healthcare facility 5. Patient information system 35 may be located outside of medical examination room 10. Patient information system 35 may be in a different location than one or more elements of healthcare facility 5. Patient information system 35 may include a patient intake system. Patient information system 35 may include a patient scheduling system.

In some embodiments, patient information system 35 may comprise a Modality Worklist (“MWL”) of healthcare facility 5. MWL is a commonly used workflow service that makes certain information available at one modality. It may be used by to manage details about scheduled examinations and patients.

MWL is part of the DICOM standard. Medical imaging systems generally support the DICOM software integration standard. Using DICOM, medical systems and software from different manufacturers can process information generated by all equipment using DICOM. The DICOM standard includes a file format definition and a network communications protocol.

The DICOM file format is a file format for medical images that includes data specific to the medical context. The DICOM file format may contain imaging information, patient information, acquisition information, and the context of the imaging study in which the image was taken. For example, a DICOM file may contain imaging information as well as information identifying the patient's name, identification number, gender, and birth date. A DICOM file may also identify the type of equipment used and various settings that the equipment was used with.

The DICOM networking protocol is a set of rules and conventions used to process and transfer DICOM-related information. The DICOM networking protocol includes commands for transferring and storing information, as well as protocols underlying searches of DICOM studies. It also includes advanced commands to aid in medical contexts, such as commands to control and follow a medical treatment, report medical staff workloads, schedule medical procedures, and others.

According to some embodiments, healthcare facility 5 includes or utilizes a medical image storage system 40. Medical image storage system 40 may be located in healthcare facility 5. In many embodiments, medical image storage system 40 is located outside of medical examination room 10. Medical image storage system 40 may be in a different location than one or more elements of healthcare facility 5.

In some embodiments, medical image storage system 40 comprises a PACS, which is a system for storing and accessing medical images from multiple devices. PACS stores images in DICOM format and may store other information in other formats. PACS may be used to store imaging information from multiple modalities, such as x-ray, MRI, and CT scans. PACS may store intravascular ultrasound imaging information. In many instances, PACS may store radiology information. Data may be stored in databases on servers, on local networks, or remotely. PACS databases may be queried using DICOM standards.

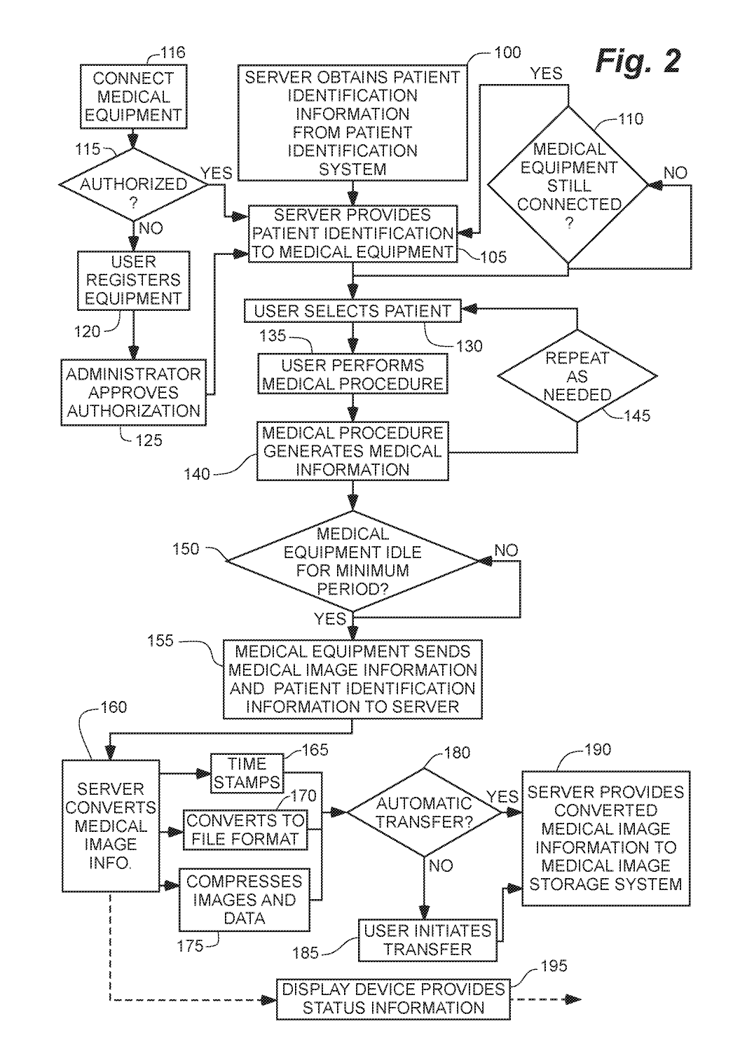

FIG. 2 is a flowchart depicting how information within an information transfer system 45 may be processed in some embodiments. Reference characters associated with process steps are provided in parentheses, while reference characters not in parentheses point to components shown in FIG. 1. Information transfer system 45 may communicate between various elements included within or utilized by healthcare facility 5. One benefit of the information transfer system 45 is to easily and automatically connect medical equipment 20 to existing information systems of healthcare facility 5. Automatic connection and communication allows for minimal required user input, fewer errors, and greater efficiency. One benefit of the system is to electronically link patient information from systems of healthcare facility 5 to information obtained from medical procedures.

In some embodiments, server 15 automatically receives patient identification information from patient information system 35 (operation 100). Patient information system 35 may comprise a MWL system. In this case, server 15 may receive the patient identification information generally available in a MWL for the scheduled procedure step. For example, when a patient first enters a healthcare facility and provides identification information at the front desk, the MWL compiles information about the patient, including any existing patient records and what medical procedure he/she is scheduled to have. Persons skilled in the art will appreciate that there are various methods for communication between the MWL and server 15. In some embodiments, the patient identification information may be accessible through the web-based interface provided by server 15. In some embodiments, the web-based interface may allow a user to assign a patient identification and/or accession number (e.g., a unique identifier for a medical procedure) to a study that has been received from an imaging device before the study is sent to the PACS for archiving.

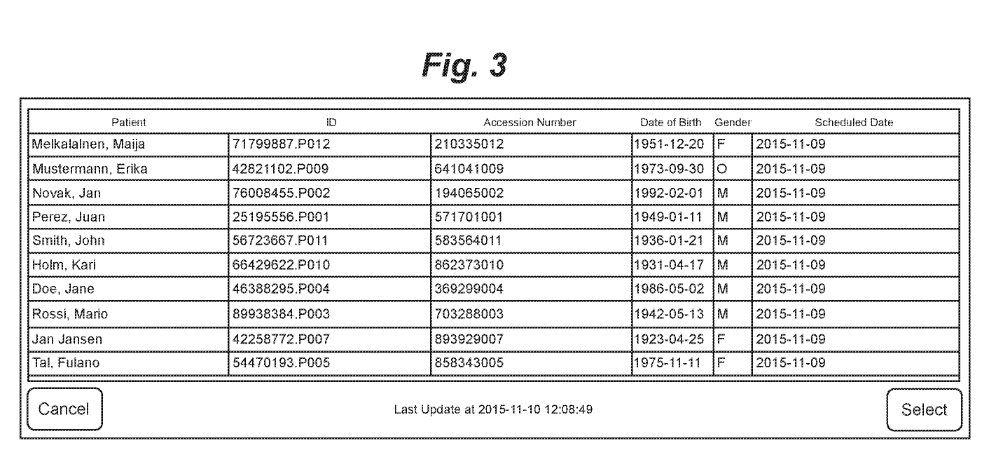

In some embodiments, patient identification information received by server 15 comprises information from a patient set. FIG. 3 provides an example of a listing of patient identification information for a patient set. According to some embodiments, a patient set comprises those patients scheduled to have a related medical procedure. For example, a patient set that ultimately makes its way to an intravascular ultrasound system may comprise those patients scheduled to have an angiography procedure, with the idea being that such patients would be more likely to need an intravascular ultrasound procedure than would the general patient population. In some instances, a patient set may comprise those patients scheduled to have a radiological procedure. In some embodiments, a patient set comprises patients scheduled to have a related medical procedure at least 12 hours prior to and up to 12 hours after the current time. In this way, the server 15 can have access to an up-to-date pool of potential candidates for a medical procedure and can ultimately provide information on those potential candidates to the medical equipment 20—in many cases, automatically. In some embodiments, server 15 may receive all patient identification information available from the MWL, but transmits only a subset of the received patient identification information to the medical equipment 20.

According to some embodiments, patient identification information can include a patient identification number. Healthcare facilities often use patient identification numbers to identify individual patients. Obtaining a patient identification number enables a healthcare technician to easily identify a patient and link the patient's records to an image or imaging study. Where DICOM systems are used, the patient identification number may include the healthcare facility's code for the patient as well as other patient ID types. Other patient ID types may include radio-frequency identifiers (“RFIDs”) or barcodes (e.g., 2-dimensional, 3-dimensional, QR codes, etc.).

In some embodiments, patient identification information may include a procedure identification number. In DICOM systems, a procedure identification number may be an accession number. A procedure identification number may assist in selection or retrieval of information. A procedure identification number may also be beneficial where a single patient has multiple examinations scheduled.

Patient identification information may include various kinds of information. FIG. 3 shows examples of data that may be included in patient identification. Patient identification information may include date of birth, gender, the date of a scheduled procedure, and the like. DICOM systems can store patient attributes such as ethnic group, persons with medical decision making authority for the patient, and other information.

In some embodiments, server 15 automatically receives patient identification information from patient information system 35 on a regular basis. Receipt of patient identification information may be automated such that no user input is required. Patient identification information may include updates of previously received information. Server 15 may automatically limit receipt of patient identification information to convenient times. Where server 15 pauses receipt of patient identification information, user input may not be required to restart receipt of patient identification information. This allows information transfer to be performed efficiently while being invisible to the user.

In some embodiments, server 15 can be configured to request patient identification information from the patient information system 35 on a periodic basis. For example, server 15 can be configured to request patient identification information from the patient information system 35 every 30 seconds, every minute, every two minutes, or at some other interval. Server 15 can be configured to inquire whether the patient information system 35 has any new patient identification information meeting predetermined criteria (e.g., a new patient just checked in and is scheduled for a related medical procedure). If the patient information system 35 has such new patient identification information, the patient information system 35 can be configured to transmit such new patient identification information to server 15. In many embodiments, all of this can occur automatically, without any involvement from a human operator.

Referring again to FIG. 2, server 15 may automatically provide patient identification information to medical equipment 20 (operation 105). In some embodiments, server 15 may automatically provide patient identification information to medical equipment 20 (operation 105) on a periodic basis. In some embodiments, a periodic basis comprises every minute, every two minutes, every five minutes, or some other interval. Server 15 may automatically limit providing patient identification information to medical equipment 20 to convenient times.

According to some embodiments, server 15 will not provide patient identification information to medical equipment 20 unless medical equipment 20 is connected to information transfer system 45 (operation 110). Connection to information transfer system 45 by medical equipment 20 may require that medical equipment 20 be connected to the computer network used by information transfer system 45. Connection to information transfer system 45 by medical equipment 20 may require that medical equipment 20 be turned on. In some cases, connection to information transfer system 45 by medical equipment 20 may require that medical equipment 20 be enabled for use. In some embodiments, server 15 can be configured to test if the medical equipment 20 is connected to information transfer system 45 on a periodic basis. For example, server 15 may be configured to check every minute, every two minutes, every five minutes, or at some other interval.

Connection to information transfer system 45 by medical equipment 20 may require that medical equipment 20 be authorized. Server 15 may provide patient identification information to medical equipment 20 automatically upon connection of medical equipment 20 to information transfer system 45 without any user input.

In some embodiments, communication with medical equipment 20 may not begin until medical equipment 20 is authorized for use (operation 115). Upon authorization of use, server 15 may automatically begin providing patient identification information and continue providing information on a periodic basis. Users may not be required to effect transfer after authorization; rather, server 15 may detect an authorized device and automatically begin provision of information. To authorize use, a user may connect medical equipment 20 to server 15 (operation 116). In some embodiments, to authorize use, a user may register the medical equipment 20 with server 15 (operation 120). Registration of medical equipment 20 may be performed using console display 25. In some embodiments, to authorize use, a user with a sufficient level of access may approve authorization (operation 125). Authorization may be approved through display device 30, 31. Sufficient levels of access may be defined differently according to the system. Information transfer system 45 may include various levels of access considered to be sufficient access levels. Examples of access levels include power users and administrators.

Console display 25 may indicate that medical equipment 20 is connected to information transfer system 45. Console display 25 may indicate connection through the colors of certain design features. A logo on console display 25 may be orange when medical equipment 20 is not connected to information transfer system 45. A logo on console display 25 may be white when medical equipment 20 is connected to information transfer system 45.

Provision of patient identification information to medical equipment 20 may facilitate selection of specific patients (operation 130). In some embodiments, selection is accomplished via console display 25. According to some embodiments, selection is accomplished using display device 30, 31.

In some embodiments, console display 25 includes a patient list. A patient list may include patient identification information automatically provided by server 15. A patient list may be displayed by clicking a button on the interface of console display 25. A patient list may be sortable by various types of patient identification information. According to some embodiments, a patient may be selected by clicking on the patient name. According to some other embodiments, a patient may be selected by highlighting the patient identification information. Accessing the selected patient's identification information as provided by the patient information system 35 can provide many advantages compared to manually entering the patient's identification information into the medical equipment 20.

Selecting a patient from the patient list can facilitate generation and/or supplementation of an imaging study. An imaging study is a test performed with a medical imaging technique. Generally, the term “imaging study” is used to refer to a single imaging procedure for a single patient. One imaging study may result in multiple images.

Upon selection of a patient, medical equipment 20 may automatically apply the selected patient identification information to a corresponding medical procedure. In some embodiments, the patient identification information is added to an open imaging study. In some embodiments, the patient identification information is displayed on console display 25. Information on the medical procedure of the selected patient may be displayed on console display 25.

Console display 25 may display information for the selected patient. Displayed information may be automatically populated upon selection of a patient. Displayed information may include patient name, date of birth, gender, a patient identification number, a procedure identification number, and the like. According to some embodiments, a user may edit and save displayed information. In some embodiments, the procedure identification number is deleted upon modifying any displayed information. In some embodiments, displayed information that has been modified must be manually reconciled with the information sent by the server 15. In some embodiments, accession (procedure identification) numbers may not be edited.

Console display 25 may display information for the medical procedure of the selected patient. Displayed information may be automatically populated upon selection of a patient. Displayed information may require input by a user. In some embodiments, a study label may be selected. A user may be able to provide a custom study label. According to some embodiments, a study location may be selected. A user may be able to provide a custom study location.

A medical procedure may be performed with medical equipment 20 (operation 135). In some embodiments, a patient may be selected prior to performing a medical procedure. In some embodiments, a patient may be selected after performing a medical procedure. The console display 25 may include a button for beginning the medical procedure.

The information transfer system 45 may be utilized with a wide variety of medical procedures that generate medical image information associated with the selected patient (operation 140). For example, the medical procedure may comprise an intravascular ultrasound procedure. Such a medical procedure may generate intravascular ultrasound image information. In some instances, the medical procedure may comprise a contrast injection procedure, which may generate a plot of quantity of contrast injected vs. time. In some instances, the medical procedure may comprise a fractional flow reserve measurement, which may generate various wave forms. In some instances, the medical procedure may comprise a radiology procedure, which may generate radiologic information such as X-ray images, computed tomography (CT) images, magnetic resonance imaging (MRI) images, ultrasound images, etc.

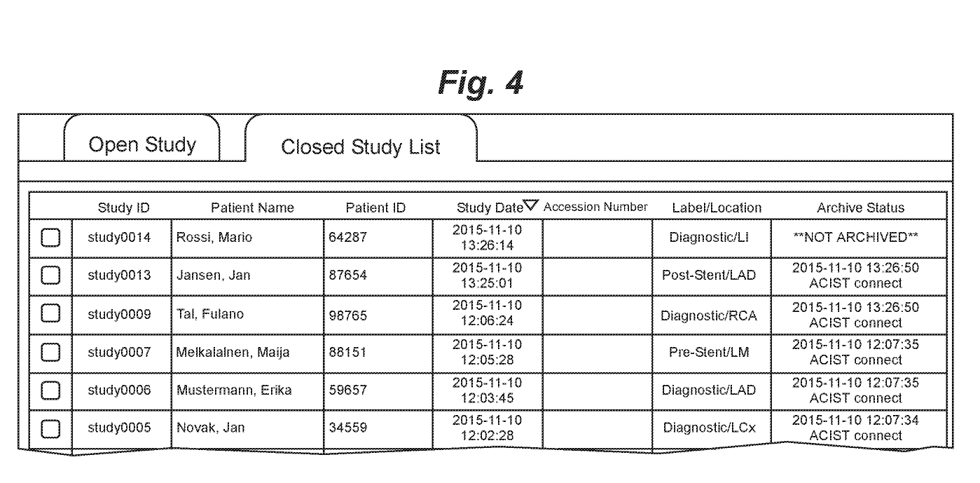

When a medical procedure has concluded, console display 25 may display medical information from the medical procedure and associated patient identification information. Console display 25 may display said medical imaging information and patient identification information in a list of concluded medical procedures (“closed study list”), as depicted in FIG. 4. A closed study list may include patient identification information provided by server 15. A closed study list may be sortable by various types of patient identification information.

Referring again to FIG. 2, after a medical procedure has concluded, a user may perform a new medical procedure. In some embodiments, a user may open a new study by selecting a new patient in the patient list (operation 145). According to some embodiments, medical imaging information and patient identification information does not need to be transferred prior to selecting a new patient. According to some other embodiments, medical imaging information and patient identification information does not need to be transferred prior to beginning a new medical procedure.

Medical equipment 20 may be configured to provide the medical imaging information and patient identification information from a selected patient and related medical procedure to the server 15 in a variety of ways. For instance, medical equipment 20 may limit providing of information to convenient times. In some embodiments, if medical equipment 20 is performing a medical procedure, medical equipment 20 automatically ceases providing information to server 15. In some embodiments, when medical equipment 20 has finished the medical procedure, medical equipment 20 automatically resumes sending information to server 15. According to some embodiments, a determination is first made whether medical equipment 20 has been inactive for a predetermined minimum period (e.g., at least one minute) (operation 150). If medical equipment 20 has been inactive for a predetermined period, transfer of information from medical equipment 20 to server 15 can begin (operation 155). In many cases, such transfer of information occurs automatically. In many cases, a user can initiate such transfer of information.

In some embodiments, users may prevent information from being transferred automatically from medical equipment 20 to server 15. For example, a user may want to perform further review of medical imaging information before storing it. In some embodiments, information transfer system 45 may include an automatic transfer option which may be enabled and disabled. Depending on the embodiment, an automatic transfer option may be enabled or disabled on console display 25 and/or on display device 30, 31.

A closed study list may include status indications reflecting progress on transferring information to the server. In some embodiments, a closed study list includes an indication where medical image information and patient identification information has not been transferred to server 15. In some embodiments, console display 25 displays the date and time when medical image information and patient identification information were transferred to server 15. After medical imaging information and patient identification information has been transferred to the medical image storage system 40, status indications in a closed study list may be automatically updated. A user may confirm the status of the medical imaging information and patient identification information by reviewing the closed study list on console display 25.

According to some embodiments, a user may add certain information to medical imaging information and personal identification information for a medical procedure using console display 25. In some embodiments, users may add measurements. Console display 25 may include measuring tools. In some embodiments, users may add annotations. Console display 25 may include annotation tools. General designs and interfaces of measurement and annotation tools will be familiar to those skilled in the art. In some other embodiments, further imaging or recording will not be available after a medical procedure has been completed.

According to some embodiments, information may be added after medical imaging information and personal identification information has been transferred to server 15. Transferred information from a closed study may be opened by clicking on patient identification information or procedure information in a closed study list. In some embodiments, where a closed study is opened, a copy of the study may be created. A copy of the study and the original study may be displayed in a grouped folder in the closed study list. In some embodiments, users may add measurements. Display device 30, 31 may include measuring tools. In some embodiments, users may add annotations. Display device 30, 31 may include annotation tools.

Where a user has opened information that has already been transferred to server 15, medical equipment 20 may not automatically transfer the updated medical imaging information and personal identification information. Updated information may be transferred by enabling automatic transfer. Updated information may be transferred by selecting a button to manually export medical imaging information and personal identification information.

In some embodiments, server 15 converts the received medical image information and patient identification information associated with a selected patient into converted medical information (operation 160). Server 15 may perform conversion automatically instead of requiring users to manually convert information. Converted medical information may include a timestamp indicating receipt of the medical image information and the patient identification information by server 15 (operation 165). Converted medical information may be converted into a specific file format (operation 170). For example, the information transferred from medical equipment 20 may be converted to a DICOM format. Converted medical image information may include compressed information (operation 175). Information may be compressed at, e.g., 60%. In some embodiments, server 15 may utilize a polar compression scheme. Compressing information at server 15 allows for shorter transfer times and network loads when transferring information to medical image storage system 40. Compressing information at server 15 also allows for smaller required spaces for storage at medical image storage system 40, by reducing the size of files and/or by reducing the need for drive space and memory devoted to compressing information at medical image storage system 40.

In many instances, converting information from the medical equipment 20 to a DICOM format at the server 15 rather than at the medical equipment 20 can provide a variety of advantages. Converted information is often larger than unconverted information. Transferring unconverted information can consume less bandwidth than transferring converted information. This can be especially important in environments in which bandwidth between the medical examination room 10 and the location of the medical image storage system 40 is scarce. Transferring the comparatively smaller unconverted information from the medical equipment 20 out of the medical examination room 10, converting the information to a DICOM format at the server 15, and then transferring the DICOM-formatted information from the server 15 to the medical image storage system 40 can make for efficient use of available bandwidth.

Server 15 may automatically provide converted information to medical image storage system 40 (operation 180). Because converted information is in proper format for storage in medical image storage system 40, server 15 may provide the converted information without additional user input. In some embodiments, server 15 may allow provision of converted information to medical image storage system 40 upon manual user request (operation 185). A manual request for server 15 to provide converted information may be made via display device 30, 31. Server 15 may provide converted information to medical image storage system 40 whether automatically or after a user manually initiates transfer (operation 190).

Display device 30, 31 may be used by different users and user types through multiple devices. In some embodiments, there may be multiple levels of users, each having a different level of access to information transfer system 45. User types may include standard users, power users, and administrators. Multiple users may be assigned each user type. To access display device 30, 31, users may need to login through an Internet browser.

Users may accomplish various tasks through display device 30, 31. Users may view overall system status. Users may view information on studies archived in medical image storage system 40. In some instances, users may resend a study to medical image storage system 40. Users may manage notifications, including email notifications. Users may authorize devices. Users may suspend and revoke devices. Users may download logs and configurations. In some instances, users may update information transfer system 45 software. Users may manage other user accounts.

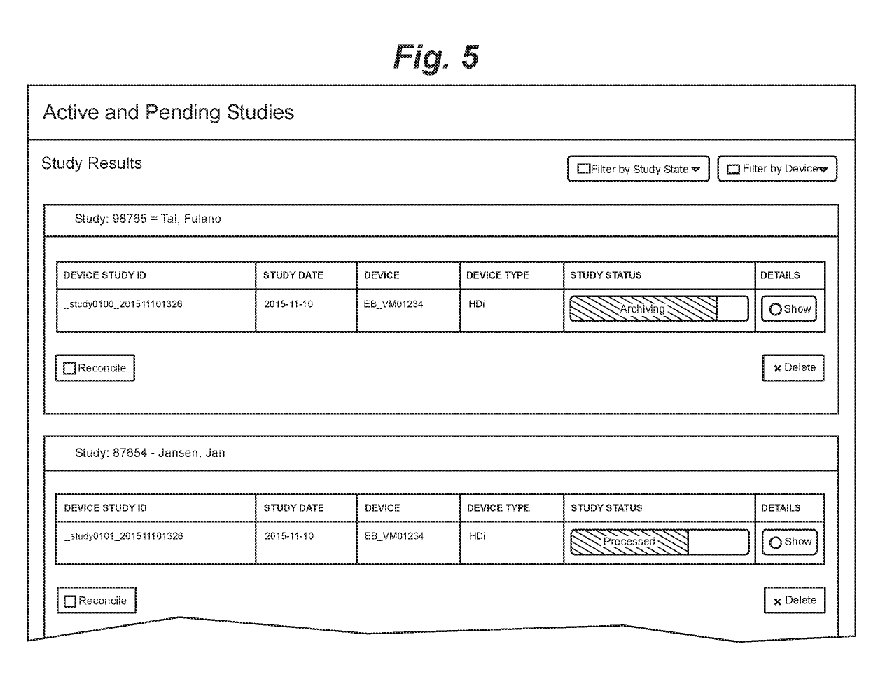

FIG. 5 depicts an example of a display that can be displayed on the display device 30, 31. In some embodiments, display device 30, 31 lists closed studies with information transferred to server 15. Display device 30, 31 may allow listed studies to be filtered and/or sorted by various options. Options may include filtering and/or sorting by stage of processing, by type of imaging device, etc.

Display device 30, 31 may include a status indication for each study on server 15 (operation 195). In some embodiments, display device 30, 31 indicates the status of information being transferred from medical equipment 20 to server 15. In some embodiments, display device 30, 31 indicates the status of information being converted on server 15. In some embodiments, display device 30, 31 indicates the status of stored information being transferred to medical image storage system 40. Display device 30, 31 may indicate progress of any transferring process as it occurs. Display device 30, 31 may indicate progress of any converting process as it occurs.

Display device 30, 31 may indicate that a closed study requires manual review. Manual review may be necessary in cases where users entered manual data in console display 25. Manual review may be necessary in cases where users modified patient identification data received in medical equipment 20. Settings may be modified to change circumstances where manual review may be required. Manual review may always be required. In some embodiments, a closed study may be manually reviewed using the web-based interface provided by server 15.

The information transfer system 45 may automatically notify users that manual review is necessary. Automatic notification may be accomplished via email and/or via icons on display device 30, 31. Automatic notification can allow users to reconcile information quickly and without performing manual analyses of information.

Display device 30, 31 may allow users to edit information associated with a study. Display device 30, 31 may allow users to edit displayed fields. Display device 30, 31 may allow users to add information. Upon completing any editing, users may click a button to save information. Saved information may be automatically converted by server 15 and transferred from server 15 to medical image storage system 40. Display device 30, 31 may allow users to delete studies that have not yet been transferred to medical image storage system 40.

In the foregoing detailed description, the invention has been described with reference to specific embodiments. However, it may be appreciated that various modifications and changes can be made without departing from the scope of the invention as set forth in the appended claims. Thus, some of the features of preferred embodiments described herein are not necessarily included in preferred embodiments of the invention which are intended for alternative uses.

1. A method comprising:
providing a server located outside of a medical examination room of a healthcare facility;
automatically receiving, at the server on a periodic basis, patient identification information from a patient information system of the healthcare facility, the patient identification information being for a set of one or more patients;
automatically providing, from the server to medical equipment, the patient identification information, thereby facilitating selection of patient identification information for a patient of the set of patients and performance of a medical procedure on the patient with the medical equipment, wherein performance of the medical procedure generates medical image information corresponding to the patient;
receiving, at the server from the medical equipment, the medical image information corresponding to the patient and the selected patient identification information for the patient;
automatically converting, at the server, the medical image information corresponding to the patient into a file that conforms to a standardized medical image file format; and
automatically providing, from the server to a medical image storage system, the file and the patient identification information for the patient.

2. The method of claim 1, wherein the patient identification information includes a patient identification number.

3. The method of claim 1, wherein automatically providing the patient identification information from the server to the medical equipment in the medical examination room occurs upon activation of the medical equipment.

4. The method of claim 1, wherein the periodic basis is at least one of: every minute, every two minutes, and every five minutes.

5. The method of claim 1, wherein the set of patients comprises one or more patients scheduled to have a related medical procedure.

6. The method of claim 5, wherein the related medical procedure comprises an angiography procedure.

7. The method of claim 5, wherein the related medical procedure comprises a radiological procedure.

8. The method of claim 5, wherein the set of patients comprises one or more patients scheduled to undergo the related medical procedure up to 12 hours prior to and up to 12 hours after the current time.

9. The method of claim 1, wherein the medical equipment includes a medical imaging device.

10. The method of claim 9, wherein the medical imaging device comprises an intravascular ultrasound system.

11. The method of claim 9, wherein the medical imaging device comprises a radiology system.

12. The method of claim 1, wherein the medical procedure comprises an intravascular ultrasound procedure, and wherein the medical image information comprises intravascular ultrasound image information.

13. The method of claim 1, wherein the medical procedure comprises a contrast injection procedure, and wherein the medical image information comprises a quantity of contrast injected into the patient vs. time.

14. The method of claim 1, wherein the medical procedure comprises a fractional flow reserve measurement, and wherein the medical image information comprises wave form information.

15. The method of claim 1, wherein the medical procedure comprises a radiology procedure, and wherein the medical image information comprises radiological information.

16. The method of claim 1, wherein the medical examination room comprises a catheterization laboratory.

17. The method of claim 1, wherein the medical examination room comprises a radiology examination room.

18. The method of claim 1, wherein automatically converting the medical image information comprises compressing the medical image information.

19. The method of claim 1, wherein the medical image storage system comprises a picture archiving and communication system.

20. The method of claim 1, further comprising:
adding a time stamp upon receipt of the medical image information and the patient identification information.

21. The method of claim 1, wherein upon detection of the medical equipment performing the medical procedure, the server ceases receiving the medical image information, and, wherein upon detection of the medical equipment finishing the medical procedure, the server resumes receiving the medical image information.

22. The method of claim 1, further comprising displaying:
a list of selected patients and associated medical procedures; and
a status of receiving the corresponding medical image information and patient identification information.

23. An information transfer system comprising:
medical equipment in a medical examination room of a healthcare facility;
a patient information system of the healthcare facility;
a medical image storage system of the healthcare facility; and
a server located outside of the medical examination room, the server configured to:
(a) automatically receive, from the patient information system, patient identification information for a set of one or more patients on a periodic basis;
(b) automatically provide, to the medical equipment, the patient identification information, thereby facilitating selection of patient identification information for a patient of the set of patients and performance of a medical procedure on the patient with the medical equipment, wherein performance of the medical procedure generates medical image information corresponding to the patient;
(c) receive, from the medical equipment, the medical image information corresponding to the patient and the selected patient identification information for the patient;
(d) automatically convert the medical image information corresponding to the patient into a file that conforms to a standardized medical image file format; and
(e) automatically provide, to the medical image storage system, the file and the patient identification information for the patient.

24. The information transfer system of claim 23, wherein the patient identification information includes a patient identification number.

25. The information transfer system of claim 23, wherein the patient information system includes a patient intake system.

26. The information transfer system of claim 23, wherein the server is configured to automatically provide the patient identification information to the medical equipment upon activation of the medical equipment.

27. The information transfer system of claim 23, wherein the periodic basis comprises at least one of: every minute, every two minutes, and every five minutes.

28. The information transfer system of claim 23, wherein the set of patients comprises one or more patients scheduled to have a related medical procedure.

29. The information transfer system of claim 28, wherein the related medical procedure comprises an angiography procedure.

30. The information transfer system of claim 28, wherein the related medical procedure comprises a radiological procedure.

31. The information transfer system of claim 28, wherein the set of patients comprises one or more patients scheduled to undergo the related medical procedure up to 12 hours prior to and up to 12 hours after the current time.

32. The information transfer system of claim 23, wherein the medical equipment includes a medical imaging device.

33. The information transfer system of claim 32, wherein the medical imaging device comprises an intravascular ultrasound system.

34. The information transfer system of claim 32, wherein the medical imaging device comprises an x-ray machine.

35. The information transfer system of claim 23, wherein the medical procedure comprises an intravascular ultrasound procedure, and wherein the medical image information comprises intravascular ultrasound image information.

36. The information transfer system of claim 23, wherein the medical procedure comprises a contrast injection procedure, and wherein the medical image information comprises a quantity of contrast injected into the patient vs. time.

37. The information transfer system of claim 23, wherein the medical procedure comprises a fractional flow reserve measurement, and wherein the medical image information comprises wave form information.

38. The information transfer system of claim 23, wherein the medical procedure comprises a radiology procedure, and wherein the medical image information comprises radiological information.

39. The information transfer system of claim 23, wherein the medical examination room comprises a catheterization laboratory.

40. The information transfer system of claim 23, wherein the medical examination room comprises a radiology examination room.

41. The information transfer system of claim 23, wherein to automatically convert the medical image information comprises to compress the medical image information.

42. The information transfer system of claim 23, wherein the medical image storage system comprises a picture archiving and communication system.

43. The information transfer system of claim 23, wherein the server is further configured to add a time stamp upon receipt of the medical image information and the patient identification information.

44. The information transfer system of claim 23, wherein the server is further configured to:
upon detection of the medical equipment performing the medical procedure, cease receiving the medical image information; and
upon detection of the medical equipment finishing the medical procedure, resume receiving the medical image information.

45. The information transfer system of claim 23, further comprising a display device, wherein the server is further configured to display on the display device:
a list of selected patients and associated medical procedures; and
a reception status for the corresponding medical image information and patient identification information.
